(19) United States
(12) Patent Application Publication
(10) Pub. No.: US 2008/0187925 A1
(43) Pub. Date: Aug. 7, 2008

(54) Biomarkers of Cancer Risk and Their Use in Cancer Detection and Prevention

(75) Inventors: Ercole Cavalieri, Waterloo, NE (US); Eleanor G. Rogan, Omaha, NE (US); Nilesh W. Gaikwad, Omaha, NE (US)

Correspondence Address: VIKSNINS HARRIS & PADYS PLLP, P.O. BOX 111098, ST. PAUL, MN, 55111-1098, US

(73) Assignee: THE BOARD OF REGENTS OF THE UNIVERSITY OF NEBRASKA, Omaha, NE (US)

(21) Appl. No.: 11/929,119

(22) Filed: Oct. 30, 2007

Related U.S. Application Data
(60) Provisional application No. 60/855,176, filed on Oct. 30, 2006.

(51) Int. Cl. C12Q 1/68; G01N 33/566

(52) U.S. Cl. 435/6; 436/501

(57) ABSTRACT

Certain embodiments of the present invention provide methods for identifying subjects that have, or are at risk for developing, cancer and for determining the effectiveness of preventive cancer treatments.

### RELATED APPLICATION(S)

This patent document claims the benefit of priority of U.S. application Ser. No. 60/855,176, filed Oct. 30, 2006, which application is herein incorporated by reference.

### STATEMENT OF GOVERNMENT SUPPORT

This invention was made with government support under U.S. Public Health Service grant P01 CA49210 from the National Cancer Institute, the U.S. Army Breast Cancer Research Program grant DAMD 17-03-1-0229 and the Avon Progress for Patients Awards Program P30 CA036727-22 from the National Institutes of Health. The government has certain rights in the invention.

### BACKGROUND

Women at high risk for breast cancer are those who are at an elevated risk for developing breast cancer as compared to the general population, e.g., those women with a Gail Model score of >1.66%. The Gail Model takes into account the following factors: age, age at menarche, age at first live birth, number of breast biopsies and history of atypical hyperplasia, number of first degree relatives with breast cancer (mother, sister and daughter), and race. A 5-year Gail Model score of >1.66% is considered high risk (Gail et al., J Natl Cancer Inst 1989; 81:1879-86). A clinical assay to identify high risk subjects, useful either in addition to or in place of determining a Gail Model score, is needed.

The development of tests, e.g., noninvasive diagnostic tests, for detecting cancer and for detecting cancer risk has been a major scientific goal for more than thirty years. The ability to test the effectiveness of a potential cancer treatment, e.g., for preventing the development or progression of cancer, is also needed.

### SUMMARY OF CERTAIN EMBODIMENTS OF THE INVENTION

Depurinating estrogen-DNA adducts found in biological samples, e.g., urine and serum samples, can be assayed as biomarkers to distinguish healthy subjects from subjects an elevated risk for cancer (e.g., breast or prostate cancer) and subjects having cancer. For example, the ratio of depurinating estrogen-DNA adducts to their respective metabolites and conjugates can serve as such a biomarker. For reasons described herein, it is important to not only determine the absolute levels of the adducts, but to also calculate the ratio of the adducts to their respective metabolites and conjugates to provide a more accurate diagnostic assay.

Accordingly, certain embodiments of the present invention provide methods for identifying a subject that is at an elevated risk for developing cancer, comprising determining the ratio of depurinating estrogen-DNA adducts to their respective metabolites and conjugates in a biological sample from the subject, wherein an increased level of the ratio, as compared to a control, indicates that the subject is at an elevated risk for developing cancer.

Certain embodiments of the present invention provide methods for identifying a subject that has cancer, comprising determining the ratio of depurinating estrogen-DNA adducts to their respective metabolites and conjugates in a biological sample from the subject, wherein an increased level of the ratio, as compared to a control, indicates that the subject has cancer.

Certain embodiments of the present invention provide methods for determining the effectiveness of a cancer preventive treatment, comprising determining the ratio of depurinating estrogen-DNA adducts to their respective metabolites and conjugates in a biological sample from the subject before and after a cancer preventive treatment, wherein a decrease in the level of the ratio after treatment indicates that the cancer treatment is effective to reduce the risk of and/or prevent the cancer.

### BRIEF DESCRIPTION OF THE FIGURES

FIG. 1 depicts the biosynthesis and metabolic activation of the estrogens, E₁ and E₂. The metabolic activation of E₁ and E₂ leads to 2- and 4-catechol derivatives, which further oxidize to yield the corresponding reactive quinones. The quinones react with DNA to form depurinating DNA adducts. In the deactivation pathway, which operates in parallel, the catechol derivatives are methylated to form methoxy catechol estrogens; in addition, the quinones are reduced by quinone reductase, as well as are conjugated with GSH, and, thus, are rendered generally harmless. As described herein, the shift in the apparent balance between these activating and deactivating pathways towards formation of depurinating DNA adducts can lead to the initiation of cancer.

FIG. 2 depicts a schematic representation of the steps carried out to purify by solid phase extraction (SPE) and analyze by ultraperformance liquid chromatography/tandem mass spectrometry (UPLC/MS-MS) the estrogen-related compounds from urine samples. The UPLC/MS-MS chromatograms of (a) 4-OHE₂, (b) 4-OHE₁, (c) 4-OCH₃E₂, (d) 4-OCH₃E₁, (e) 4-OHE₂-1-N7Gua, (f) 4-OHE₁-1-N7Gua, (g) 4-OHE₂-1-N3Ade and (h) 4-OHE₁-1-N3Ade that are shown in the figure are representatives from the 40 different estrogen-related compounds seen in the urine samples.

FIG. 3 depicts SPE recovery of the standard 40 estrogen-related compounds. The 2 ml aliquots of activated charcoal-treated human urine samples were spiked with the total (A) 250, (B) 500 and (C) 1000 pg of 40 estrogen-related compounds before and after (control) passing over phenyl SPE cartridges. The recovery of each compound was determined by comparing the experimental values to the controls.

FIG. 4 depicts depurinating estrogen-DNA adducts in the urine of healthy women, high-risk women and women with breast cancer. The ordinate of this bar graph corresponds to the ratio of depurinating DNA adducts divided by their respective estrogen metabolites and conjugates:

$$\frac{\begin{matrix}4 \text{-} {OHE}_{1} ( {E}_{2} ) \text{-} 1 \text{-} N 3 Ade + \\ 4 \text{-} {OHE}_{1} ( {E}_{2} ) \text{-} 1 \text{-} N 7 Gua\end{matrix}}{\begin{matrix}4 \text{-} catechol estrogens + \\ 4 \text{-} catechol estrogen conjugates\end{matrix}} + \frac{2 \text{-} {OHE}_{1} ( {E}_{2} ) \text{-} 6 \text{-} N 3 Ade}{\begin{matrix}2 \text{-} catechol estrogens + \\ 2 \text{-} catechol estrogen conjugates\end{matrix}} \times 1000.$$

The mean sum of the ratios for control women was significantly lower than those for the high-risk women (p<0.001) and women with breast cancer (p<0.001). The mean sums of the ratios for high-risk women and women with breast cancer were not significantly different (p=0.62). ^{†}These are two urine samples from the same subject, collected 11 weeks apart. Statistical calculations used one average value for this subject.

### DETAILED DESCRIPTION

HPLC was used to analyze depurinating estrogen-DNA adducts, estrogen metabolites and estrogen conjugates in urine samples. As described herein, the ratio of the adducts to their corresponding metabolites and conjugates provides a biomarker that can be used to distinguish women to be at high risk of developing breast cancer (e.g., those having a Gail Model score>1.66%; Gail et al., J Natl Cancer Inst 1989; 81:1879-86) and those with breast cancer from healthy control women. The development of such an assay will be invaluable in assessing cancer risk and response to preventive treatment.

Accordingly, certain embodiments of the present invention provide methods for identifying a subject that has cancer, or is at an elevated risk for developing cancer, comprising determining the ratio of depurinating estrogen-DNA adducts to their respective metabolites and conjugates in a biological sample from the subject, wherein an increased level of the ratio, as compared to a control, indicates that the subject has cancer, or is at an elevated risk for developing cancer.

Certain embodiments of the present invention provide methods for determining the effectiveness of a preventive cancer treatment, comprising determining the ratio of depurinating estrogen-DNA adducts to their respective metabolites and conjugates in a biological sample from the subject before and after a preventive cancer treatment, wherein a decrease in the level of the ratio after treatment indicates that the cancer treatment is effective to reduce the risk of (e.g., prevent) cancer.

In certain embodiments of the invention, the methods further comprise purifying the adducts, metabolites and conjugates from the sample using solid phase extraction (SPE).

In certain embodiments of the invention, the methods further comprise analyzing the adducts, metabolites and conjugates utilizing ultraperformance liquid chromatography/tandem mass spectrometry (UPLC/MS-MS).

In certain embodiments of the invention, the methods further comprise administering to those subjects that have or are at risk for developing cancer a therapy effective to reduce the risk of (e.g., prevent) cancer.

In certain embodiments of the invention, the therapy is effective to decrease the ratio of depurinating estrogen-DNA adducts to their respective metabolites and conjugates.

In certain embodiments of the invention, the cancer is breast cancer or prostate cancer.

In certain embodiments of the invention, the cancer is breast cancer, prostate cancer, leukemia, non-Hodgkins lymphoma, colon cancer, pancreatic cancer, lung cancer (e.g., lung cancer of non-smokers), ovarian cancer, endometrial cancer, testicular cancer, kidney cancer, or brain cancer.

In certain embodiments of the invention, the sample is breast tissue, prostate tissue, urine, serum, sputum, or nipple aspirate fluid.

In certain embodiments of the invention, the sample is breast tissue, prostate tissue, urine, serum, sputum, nipple aspirate fluid, colon tissue, pancreas tissue, lung tissue, ovary tissue, endometrium, testis tissue, kidney tissue, or brain tissue.

In certain embodiments of the invention, the subject has cancer.

In certain embodiments of the invention, the subject is at an elevated risk for developing cancer.

In certain embodiments of the invention, an adduct is an N3Ade adduct of 4-OHE₁(E₂).

In certain embodiments of the invention, an adduct is an N7Gua adduct of 4-OHE₁(E₂).

In certain embodiments of the invention, the subject is a male.

In certain embodiments of the invention, the subject is a female.

Estrogens can become endogenous carcinogens via formation of catechol estrogen quinones, which react with DNA to form specific depurinating estrogen-DNA adducts. The mutations resulting from these adducts can lead to cell transformation and the initiation of cancer, e.g., breast cancer or prostate cancer.

Estrogen metabolites, conjugates and depurinating DNA adducts in urine samples from healthy control women, high-risk women, and women with breast cancer were analyzed. The estrogen metabolites, conjugates and depurinating DNA adducts were identified and quantified using UPLC/MS-MS. The levels of the ratios of depurinating DNA adducts to their respective estrogen metabolites and conjugates were significantly higher in high-risk women and women with breast cancer than in control subjects. The high-risk and breast cancer groups were not significantly different. After adjusting for patient characteristics, these ratios were still significantly associated with health status. Thus, the ratios of depurinating DNA adducts to their respective estrogen metabolites and conjugates are useful for early detection of breast cancer risk and for determining response to treatment, e.g., preventive treatment.

As used herein, the terms “treat” and “treatment” can refer to therapeutic treatment and prophylactic or preventative treatment. In some embodiments of the invention, the object is to prevent or decrease the development of cancer. Those subjects in need of treatment include those having a predisposition to developing cancer. Accordingly, certain embodiments of the invention relate to determining the effectiveness of a cancer treatment.

The terms “cancer” and “cancerous” refer to the physiological condition in mammals that is typically characterized by unregulated cell growth. Examples of cancers include, but are not limited to, breast cancer and prostate cancer.

Some abbreviations used herein: Cys, cysteine; ESI, electrospray ionization; E₁(E₂)-Q, estrone(estradiol)-quinones; GSH, glutathione; 4-OHE₂, 4-hydroxyestradiol; 4-OHE₁(E₂)-1-N3Ade, 4-hydroxyestrone(estradiol)-1-N3Adenine; 4-OHE₁(E₂)-1-N7Gua, 4-hydroxyestrone(estradiol)-1-N7Guanine; 2-OHE₁(E₂)-6-N3Ade, 2-hydroxyestrone(estradiol)-6-N3Adenine; MRM, multiple reaction monitoring; NAcCys, N-acetylcysteine; NI, negative ion; PI, positive ion; SPE, solid-phase extraction; UPLC/MS-MS, ultraperformance liquid chromatography/tandem mass spectrometry.

The invention will now be illustrated by the following non-limiting Examples.

### Example 1

### Biomarkers of Breast Cancer Risk

The development of tests, e.g., noninvasive tests, for breast cancer risk has been a major goal for more than thirty years. As described herein, biomarkers of risk have been identified that are related to what is believed to be the first critical step in the initiation of breast cancer, namely, the reaction of catechol estrogen quinone metabolites with DNA (Cavalieri et al., Biochim Biophys Acta 2006; 1766:63-78).

Exposure to estrogens is a risk factor for the development of breast cancer. Specific oxidative metabolites of estrogens, namely, catechol estrogen quinones, can react with DNA. These metabolites may become endogenous chemical carcinogens. Mutations thought to be generated by this DNA damage may result in the initiation of cancer (Cavalieri et al., Biochim Biophys Acta 2006; 1766:63-78; and Cavalieri et al., PNAS 1997; 94:10937-42).

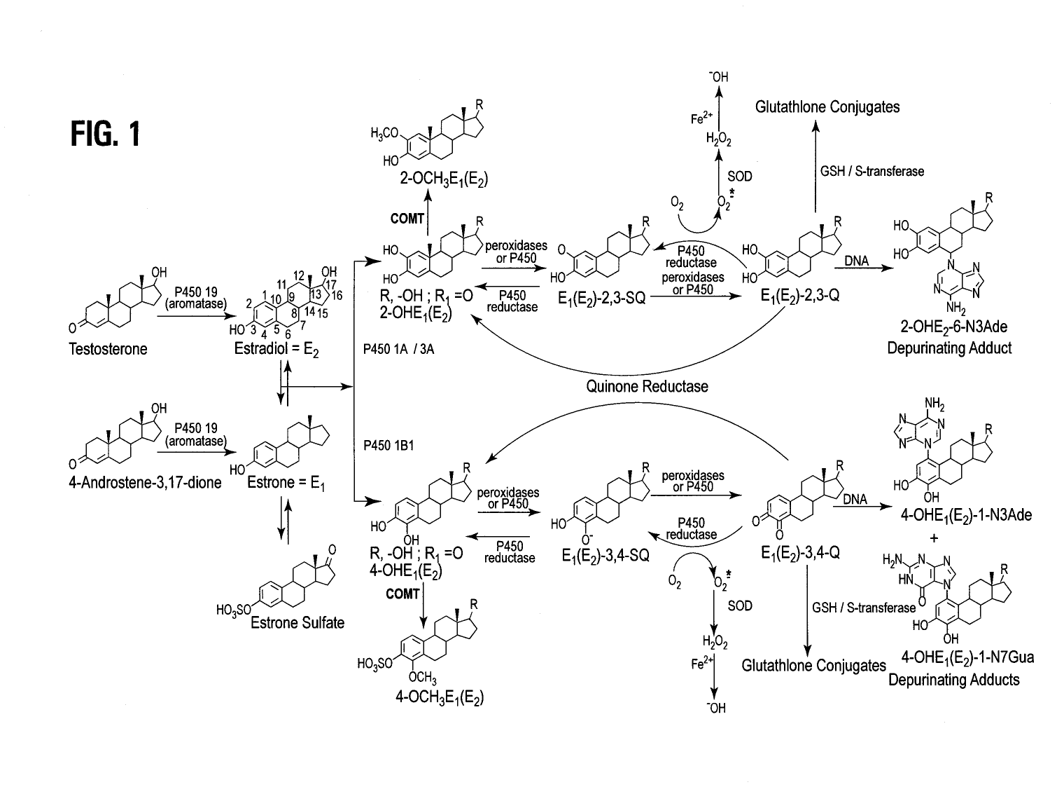

As illustrated in FIG. 1, in the metabolism of estrogens, there are activating pathways (Hayes et al., PNAS 1996; 93:9776-81 and Zahid et al, Free Radic Biol Med. 2007; 43:1534-1540) that lead to the formation of the estrogen quinones, estrone (estradiol) quinones [E₁(E₂)-Q], which can react with DNA. There are also deactivating pathways that limit formation of the quinones and/or prevent their reaction with DNA. These include the methylation of catechol estrogens (Mannisto et al., Pharmacol Rev 1999; 51:593-628), conjugation of the E₁(E₂)-Q with GSH (Cavalieri et al., Chem Res Toxicol 2001; 14:1041-1050) and reduction of the quinones to catechols (Gaikwad et al., Free Radic Biol Med 2007; 43:1289-1298).

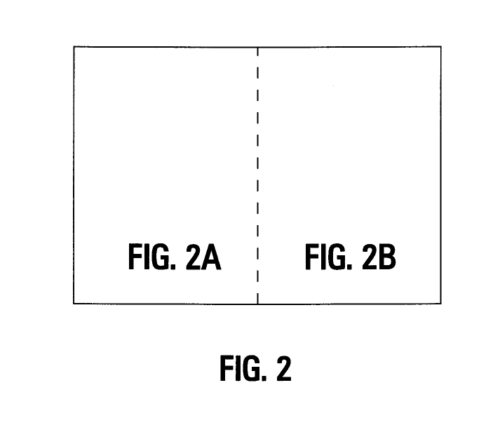

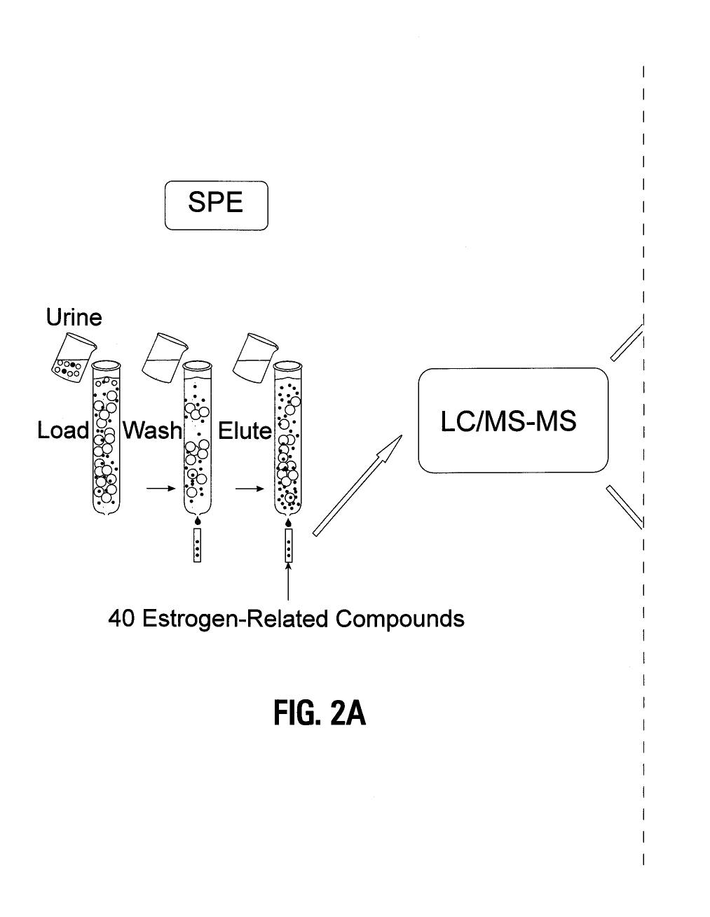

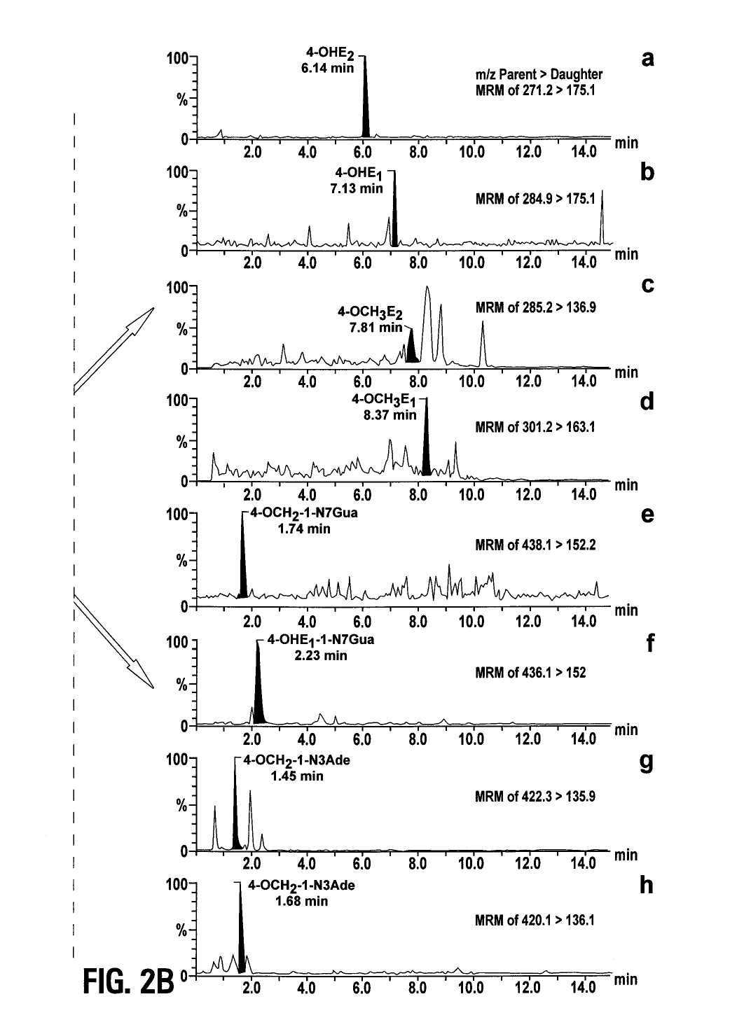

When E₁(E₂)-3,4-Q react with DNA, they form predominantly the depurinating adducts 4-hydroxyestrone(estradiol)-1-N3Adenine [4-OHE₁(E₂)-1-N3Ade] and 4-hydroxyestrone(estradiol)-1-N7Guanine [4-OHE₁(E₂)-1-N7Gua], whereas E₁(E₂)-2,3-Q form much lower levels of 2-hydroxyestrone(estradiol)-6-N3Adenine [2-OHE₁(E₂)-6-N3Ade] (see FIGS. 1 and 2) (Zahid et al., Chem Res Toxicol 2006; 19:164-72). Both E₁(E₂)-3,4-Q and E₁(E₂)-2,3-Q form much lower levels of stable DNA adducts than depurinating adducts (Cavalieri et al., PNAS 1997; 94:10937-42; Li et al., Carcinogenesis 2004; 25:289-97; and Zahid et al., Chem Res Toxicol 2006; 19:164-72). Once released from the DNA, the depurinating estrogen-DNA adducts are shed from cells into the bloodstream and, eventually, are excreted in urine. The release of the depurinating adducts generates apurinic sites in DNA, which in turn, are thought to induce mutations that can lead to the development of cancer.

As described herein, initiation of cancer by estrogens is based on estrogen metabolism in which the homeostatic balance between activating and deactivating pathways is disrupted (see FIG. 1). Activating pathways oxidize E₁ and E₂ to their catechol estrogen quinones, whereas the deactivating pathways block oxidation. A variety of factors, such as diet, environment and lifestyle, can unbalance the equilibrium between these two pathways. When estrogen metabolism is balanced, the level of estrogen-DNA adducts in tissue and urine is low and/or the levels of estrogen metabolites and conjugates high. In contrast, when estrogen metabolism is unbalanced, the level of DNA adducts in tissue and urine is high and/or the levels of estrogen metabolites and conjugates are low. As described herein, it is this imbalance in estrogen metabolism, leading to relatively high levels of estrogen-DNA adducts, that is thought to be a critical determinant of cancer initiation.

It was hypothesized that estrogen metabolites, conjugates and depurinating DNA adducts would differ between healthy women and women with breast cancer or at high risk of breast cancer. To test this hypothesis, a cross-sectional study was conducted in which 40 estrogen metabolites, conjugates and depurinating DNA adducts were analyzed in urine samples from healthy women, women at high risk for breast cancer based on Gail Model score>1.66%, and women with breast carcinoma. In certain embodiments of the invention, all 40 compounds are analyzed so as to determine the ratio. In certain embodiments of the invention, not all 40 compounds are analyzed so as to determine the ratio (e.g., the ratio may be determined using any combination of the compounds).

Analysis of urine samples. Following partial purification of the urine samples by SPE (FIG. 2), the 40 estrogen-related compounds were analyzed using UPLC/MS-MS. An advantage of having the MS detector in MRM mode over conventional analysis is that fragmentation of the molecules could be used to specifically and separately identify each of the estrogen-related compounds without interference from impurities or unwanted components (FIG. 2). Each metabolite was detected and identified based on unique parameters, such as mass (parent and daughter), retention time, ionization mode (positive and negative) (see Table 1). The typical spectra of representative estrogen derivatives, which were obtained in a single injection, are shown in FIG. 2. The levels of estrogen-related compounds for a high risk woman, measured from single injection, are presented in Table 2.

TABLE 1
Mass spectrometric parameters.*

| | | | ESI | Parent | Daughters | Cone | | Retention | LOD |
|---|---|---|---|---|---|---|---|---|---|
| No. | Compound | Mass | Mode | m/z | m/z | volt | Collision | time | (fmol) |
| 1 | AD | 286.2 | Pos. | 287.1 | 97.1 | 40 | 19 | 8.43 | 14 |
| 2 | Test | 288.2 | Pos. | 289.2 | 97.0 | 40 | 19 | 7.97 | 35 |
| 3 | E₁-Sulfate | 350.1 | Neg. | 249.0 | 269.0 | 50 | 28 | 6.61 | 143 |
| 4 | E₂ | 272.4 | Pos. | 273.2 | 135.2 | 30 | 14 | 7.74 | 184 |
| 5 | E₁ | 270.1 | Pos. | 271.2 | 253.2 | 25 | 14 | 8.43 | 148 |
| 6 | 2-OHE₂ | 288.2 | Pos. | 271.2 | 175.1 | 30 | 14 | 6.74 | 69 |
| 7 | 2-OHE₁ | 286.2 | Neg. | 285.0 | 160.9 | 65 | 37 | 7.3 | 18 |
| 8 | 4-OHE₂ | 288.2 | Pos. | 271.2 | 175.1 | 30 | 14 | 6.14 | 69 |
| 9 | 4-OHE₁ | 286.2 | Neg. | 284.9 | 161.0, | 65 | 35 | 7.13 | 35 |
| | | | | | 175.1 | | | | |
| 10 | 16α-E₂ | 288.4 | Pos. | 289.0 | 107.0 | 25 | 14 | 2.42 | 867 |
| 11 | 16α-E₁ | 286.4 | Neg. | 285.1 | 145.1 | 30 | 15 | 4.67 | 349 |
| 12 | 2-OCH₃E₂ | 302.2 | Pos. | 285.2 | 136.9, | 32 | 15 | 8.25 | 330 |
| | | | | | 189.1 | | | | |
| 13 | 2-OCH₃E₁ | 300.2 | Pos. | 301.2 | 136.9, | 30 | 17 | 8.85 | 333 |
| | | | | | 163.1 | | | | |
| 14 | 4-OCH₃E₂ | 302.2 | Pos. | 285.2 | 136.9, | 32 | 15 | 7.81 | 66 |
| | | | | | 189.1 | | | | |
| 15 | 4-OCH₃E₁ | 300.2 | Pos. | 301.2 | 163.1, | 30 | 17 | 8.37 | 133 |
| | | | | | 283.1 | | | | |
| 16 | 2-OH—OCH₃E₂ | 302.4 | Pos. | 285.2 | 189.1 | 32 | 15 | 8.71 | 165 |
| 17 | 2-OH—OCH₃E₁ | 300.4 | Pos. | 301.2 | 163.1 | 30 | 17 | 9.07 | 33 |
| 18 | 2-OHE₂-1-SG | 593.7 | Pos. | 594.1 | 319.1, | 42 | 20 | 1.72 | 8.4 |
| | | | | | 465.0 | | | | |
| 19 | 2-OHE₂-4-SG | 593.7 | Pos. | 594.0 | 319.1, | 35 | 21 | 2.32 | 8.4 |
| | | | | | 465.4 | | | | |
| 20 | 2-OHE₁-1-SG | 591.0 | Pos. | 592.1 | 316.8 | 45 | 22 | 2.65 | 1.7 |
| 21 | 2-OHE₁-4-SG | 591.0 | Pos. | 592.2 | 317.1, | 40 | 22 | 2.65 | 1.7 |
| | | | | | 463.2 | | | | |
| 22 | 2-OHE₂-1 + 4-Cys | 407.2 | Pos. | 408.2 | 319.0 | 30 | 17 | 1.73 | 12 |
| 23 | 2-OHE₁-1-Cys | 405.2 | Pos. | 406.0 | 316.9 | 35 | 15 | 3.25 | 6.2 |
| 24 | 2-OHE₁-4-Cys | 405.2 | Pos. | 406.2 | 317.1 | 30 | 17 | 3.25 | 6.2 |
| 25 | 2-OHE₂-1-NAcCys | 449.2 | Pos. | 450.1 | 162.0, | 25 | 14 | 4.07 | 5.6 |
| | | | | | 287.4 | | | | |
| 26 | 2-OHE₂-4-NAcCys | 449.2 | Pos. | 450.2 | 162.0, | 30 | 14 | 4.07 | 5.6 |
| | | | | | 287.2 | | | | |
| 27 | 2-OHE₁-1-NAcCys | 447.2 | Pos. | 448.1 | 162.0, | 30 | 13 | 6.05 | 5.6 |
| | | | | | 285.4 | | | | |
| 28 | 2-OHE₁-4-NAcCys | 447.2 | Pos. | 448.0 | 162.0, | 35 | 14 | 6.05 | 5.6 |
| | | | | | 284.9 | | | | |
| 29 | 4-OHE₂-2-SG | 593.2 | Pos. | 594.4 | 318.9, | 42 | 20 | 2.33 | 8.4 |
| | | | | | 464.8 | | | | |
| 30 | 4-OHE₁-2-SG | 591.2 | Pos. | 592.3 | 317.1, | 45 | 22 | 2.65 | 8.5 |
| | | | | | 462.9 | | | | |
| 31 | 4-OHE₂-2-Cys | 407.2 | Pos. | 408.0 | 318.9, | 40 | 16 | 2.24 | 2.4 |
| | | | | | 286.1 | | | | |
| 32 | 4-OHE₁-2-Cys | 405.2 | Pos. | 406.0 | 316.9, | 35 | 15 | 2.84 | 6.2 |
| | | | | | 389.0 | | | | |
| 33 | 4-OHE₂-2-NAcCys | 449.2 | Pos. | 450.1 | 162.1 | 35 | 15 | 5.91 | 5.6 |
| 34 | 4-OHE₁-2-NAcCys | 447.2 | Pos. | 448.3 | 161.8 | 35 | 14 | 6.64 | 2.2 |
| 35 | 4-OHE₂-1-N7Gua | 437.2 | Pos. | 438.1 | 152.2, | 62 | 38 | 1.74 | 2.3 |
| | | | | | 272.0 | | | | |
| 36 | 4-OHE₁-1-N7Gua | 435.2 | Pos. | 436.1 | 152.0, | 62 | 38 | 2.23 | 2.2 |
| | | | | | 271.9 | | | | |
| 37 | 4-OHE₂-1-N3Ade | 421.2 | Pos. | 422.3 | 135.9, | 62 | 45 | 1.45 | 5.9 |
| | | | | | 257.1 | | | | |
| 38 | 4-OHE₁-1-N3Ade | 419.2 | Pos. | 420.1 | 296.0, | 60 | 44 | 1.68 | 2.4 |
| | | | | | 136.1 | | | | |
| 39 | 2-OHE₂-6-N3Ade | 421.1 | Pos. | 422.2 | 136.0, | 26 | 10 | 1.05 | 1.2 |
| | | | | | 287.0 | | | | |
| 40 | 2-OHE₁-6-N3Ade | 419.1 | Pos. | 420.0 | 135.9 | 26 | 10 | 1.41 | 2.4 |

*List of the 40 estrogen-related compounds with the masses of parent and daughter ions and the ionization mode that were used for MRM method optimization. The last column indicates the limit of detection for each compound.

TABLE 2
Representative metabolic profile of a urine sample obtained
from a high risk woman.¹

| | | pmole/mg | Total |
|---|---|---|---|
| | | creatinine | pmole/mg |
| NO. | Compound | mean, n = 2 | creatinine |
| 1 | Androstenedione | 1.56 | 1.56 |
| 2 | Testosterone | 0.24 | 0.24 |
| 3 | E₁ Sulfate | 1.81 | 1.81 |
| 4 | E₂* | 5.29 | 15.93 |
| 5 | E₁* | 10.64 | |
| 6 | 2-OHE₂ | 3.09 | 3.15 |
| 7 | 2-OHE₁ | 0.05 | |
| 8 | 4-OHE₂ | 2.64 | 2.91 |
| 9 | 4-OHE₁ | 0.27 | |
| 10 | 16α-OHE₂ | 12.12 | 38.64 |
| 11 | 16α-OHE₁ | 26.52 | |
| 12 | 2-OCH₃E₂ | 1.95 | 49.81 |
| 13 | 2-OHC₃E₁ | 47.87 | |
| 14 | 4-OCH₃E₂ | 0.41 | 5.08 |
| 15 | 4-OCH₃E₁ | 4.67 | |
| 16 | 2-OH-3-OCH₃E₂ | 1.91 | 10.27 |
| 17 | 2-OH-3-OCH₃E₁ | 8.36 | |
| 18 | 2-OHE₂-1-SG | 0.17 | 3.10 |
| 19 | 2-OHE₂-4-SG | 0.17 | |
| 20 | 2-OHE₁-1-SG | 0.49 | |
| 21 | 2-OHE₁-4-SG | 0.47 | |
| 22 | 2-OHE₂-1+4-Cys | 0.27 | |
| 23 | 2-OHE₁-1-Cys | 0.10 | |
| 24 | 2-OHE₁-4-Cys | 0.44 | |
| 25 | 2-OHE₂-1-NAcCys | 0.07 | |
| 26 | 2-OHE₂-4-NAcCys | 0.07 | |
| 27 | 2-OHE₁-1-NAcCys | 0.43 | |
| 28 | 2-OHE₁-4-NAcCys | 0.43 | |
| 29 | 4-OHE₂-2-SG | 0.51 | 1.77 |
| 30 | 4-OHE₁-2-SG | 0.50 | |
| 31 | 4-OHE₂-2-Cys | 0.13 | |
| 32 | 4-OHE₁-2-Cys | 0.06 | |
| 33 | 4-OHE₂-2-NAcCys | 0.29 | |
| 34 | 4-OHE₁-2-NAcCys | 0.28 | |
| 35 | 4-OHE₂-1-N7Gua | 0.48 | 2.81 |
| 36 | 4-OHE₁-1-N7Gua | 2.33 | |
| 37 | 4-OHE₂-1-N3Ade | 137.78 | 137.90 |
| 38 | 4-OHE₁-1-N3Ade | 0.13 | |
| 39 | 2-OHE₂-6-N3Ade | 0.06 | 0.07 |
| 40 | 2-OHE₁-6-N3Ade | 0.02 | |

| | |
|---|---|
| (Ratio-4)² × 1000 | 935 |
| (Ratio-2)³ × 1000 | 1 |
| (Ratio-4) + (Ratio-2) × 1000 | 936 |

¹Typically, each 2-ml urine sample was analyzed at least 2 times.
The data obtained from LC/MS-MS were processed and normalized to
creatinine levels. Since the E₁ and E₂ derivatives are interconvertible,
the total amount for each E₁ plus E₂ derivative in the various categories are
presented in the last column and used for calculating the final ratios of
depurinating adducts to the respective metabolites and conjugates.
${ }^{2} \frac{4 \text{-} {OHE}_{1} ( {E}_{2} ) \text{-} 1 \text{-} N 3 Ade + 4 \text{-} {OHE}_{1} ( {E}_{2} ) \text{-} 1 \text{-} N 7 Gua}{4 \text{-} catechol estrogens + 4 \text{-} catechol estrogen conjugates} = \\ \frac{\#37 + 38 + 35 + 36}{\#8 + 9 + 14 + 15 + 29 through 34} {\\ }^{3} \frac{2 \text{-} {OHE}_{1} ( {E}_{2} ) \text{-} 6 \text{-} N 3 Ade}{2 \text{-} catechol estrogens + 2 \text{-} catechol estrogen conjugates} = \\ \frac{\#39 + 40}{\#6 + 7 + 12 + 13 + 16 through 28}$
*Free E₂ and E₁ in the urine sample.

TABLE 3
Urinary levels of estrogen compounds in healthy women, high-risk women and women with breast cancer

| | | | | | | | Breast Cancer | | |
|---|---|---|---|---|---|---|---|---|---|
| | | Control (n = 46) | | High Risk (n = 12) | | | | Min- | (n = 18) |
| No. | Compound | Median | Min-Max | Median | Min-Max | p-value* | Median | Max | p-value** |
| 1 | AD | 9.9 | 2.1-108 | 4.2 | 1.3-11.5 | 0.003*** | 5.5 | 0.4-95.1 | 0.047 |
| 2 | Test | 2.2 | 0.2-16.5 | 0.8 | 0.2-2.8 | 0.008 | 1.1 | 0.5-3.7 | 0.050 |
| 3 | E₁-Sulfate | 5.0 | 0.1-382 | 2.4 | 0.1-10.6 | 0.087 | 1.1 | 0.1-121 | 0.032 |
| 4 | E₂ | 31.7 | 9.1-3865 | 11.4 | 4.7-80.0 | 0.007 | 28.1 | 3.6-151 | 0.943 |
| 5 | E₁ | | | | | | | | |
| 6 | 2-OHE₂ | 10.4 | 1.7-564 | 7.3 | 1.6-26.5 | 0.035 | 5.6 | 0.0-38.8 | 0.006 |
| 7 | 2-OHE₁ | | | | | | | | |
| 8 | 4-OHE₂ | 12.4 | 2.4-157 | 8.1 | 2.9-43.3 | 0.138 | 5.2 | 0.0-28.0 | 0.008 |
| 9 | 4-OHE₁ | | | | | | | | |
| 10 | 16α-E₂ | 168 | 10.3-638 | 33.7 | 0.0-279 | 0.001 | 10.9 | 0.0-86.3 | <0.001 |
| 11 | 16α-E₁ | | | | | | | | |
| 12 | 2-OCH₃E₂ | 49.7 | 4.7-568 | 31.1 | 6.5-452 | 0.275 | 26.8 | 2.2-171 | 0.044 |
| 13 | E₁2-OCH₃ | | | | | | | | |
| 14 | 4-OCH₃E₂ | 73.1 | 12.6-3979 | 5.9 | 1.6-37.1 | <0.001 | 21.5 | 4.5-53.2 | <0.001 |
| 15 | 4-OCH₃E₁ | | | | | | | | |
| 16 | 2-OH—OCH₃E₂ | | | | | | | | |
| 17 | 2-OH—OCH₃E₁ | | | | | | | | |
| 18 | 2-OHE₂-1-SG | 11.2 | 0.8-79.8 | 4.6 | 1.2-18.7 | 0.005 | 3.1 | 1.1-16.9 | 0.001 |
| 19 | 2-OHE₂-4-SG | | | | | | | | |
| 20 | 2-OHE₁-1-SG | | | | | | | | |
| 21 | 2-OHE₁-4-SG | | | | | | | | |
| 22 | 2-OHE₂-1 + 4-Cys | | | | | | | | |
| 23 | 2-OHE₁-1-Cys | | | | | | | | |
| 24 | 2-OHE₁-4-Cys | | | | | | | | |
| 25 | 2-OHE₂-1-NAcCys | | | | | | | | |
| 26 | 2-OHE₂-4-NAcCys | | | | | | | | |
| 27 | 2-OHE₁-1-NAcCys | | | | | | | | |
| 28 | 2-OHE₁-4-NAcCys | | | | | | | | |
| 29 | 4-OHE₂-2-SG | 2.7 | 0.7-24.6 | 1.4 | 0.6-6.8 | 0.032 | 1.3 | 0.4-8.9 | 0.027 |
| 30 | 4-OHE₁-2-SG | | | | | | | | |
| 31 | 4-OHE₂-2-Cys | | | | | | | | |
| 32 | 4-OHE₁-2-Cys | | | | | | | | |
| 33 | 4-OHE₂-2-NAcCys | | | | | | | | |
| 34 | 4-OHE₁-2-NAcCys | | | | | | | | |
| 35 | 4-OHE₂-1-N7Gua | 0.7 | 0.0-4.8 | 1.2 | 0.2-2106 | 0.238 | 1.6 | 0.4-11.8 | 0.007 |
| 36 | 4-OHE₁-1-N7Gua | | | | | | | | |
| 37 | 4-OHE₂-1-N3Ade | 0.7 | 0.0-18.8 | 1.8 | 0.5-138 | 0.007 | 1.2 | 0.1-288 | 0.085 |
| 38 | 4-OHE₁-1-N3Ade | | | | | | | | |
| 39 | 2-OHE₂-6-N3Ade | 0.1 | 0.0-6.5 | 0.1 | 0.0-0.7 | 0.999 | 0.1 | 0.0-5.4 | 0.960 |
| 40 | 2-OHE₁-6-N3Ade | | | | | | | | |

*Bonferroni-adjusted p-value for comparing control vs. high risk using Mann-Whitney test.
**Bonferroni-adjusted p-value for comparing control vs. breast cancer by using Mann-Whitney test.
***Significant p-values are shown in bold.

Treatment of the urine with glucuronidase/sulfatase led to significant increases (10-20 fold) in the levels of E₁ and E₂, while the levels of estrogen metabolites, conjugates and adducts changed marginally, and in many cases decreased because of the incubation for 8 hours at 37° C. To avoid potential artifacts and errors that could be introduced by maintaining the urine samples at 37° C. for 8 hours, all the analyses were carried out without glucuronidase/sulfatase treatment. Therefore, the observed levels of E₁ and E₂, as reported in Table 2, for example, were 10-20 fold lower than the total values. Since estrone and estradiol are constantly inter-converting, estrone and estradiol values were combined for the derivatives (Tables 2 and 3). The GSH conjugates of estrogen quinones are further converted to Cys and NAcCys conjugates via the mercapturic acid biosynthesis pathway (Boyland et al., Adv Enzymol Relat Areas Mol Biol 1969; 32:173-219). Hence the values of 2-conjugates and 4-conjugates (Tables 2 and 3) were combined, which reflects the total protection by GSH from 2- or 4-quinones, respectively. The results presented here clearly demonstrate the ability of SPE combined with UPLC/MS-MS analysis to resolve, identify and quantify 40 estrogen-related compounds with accuracy and speed.

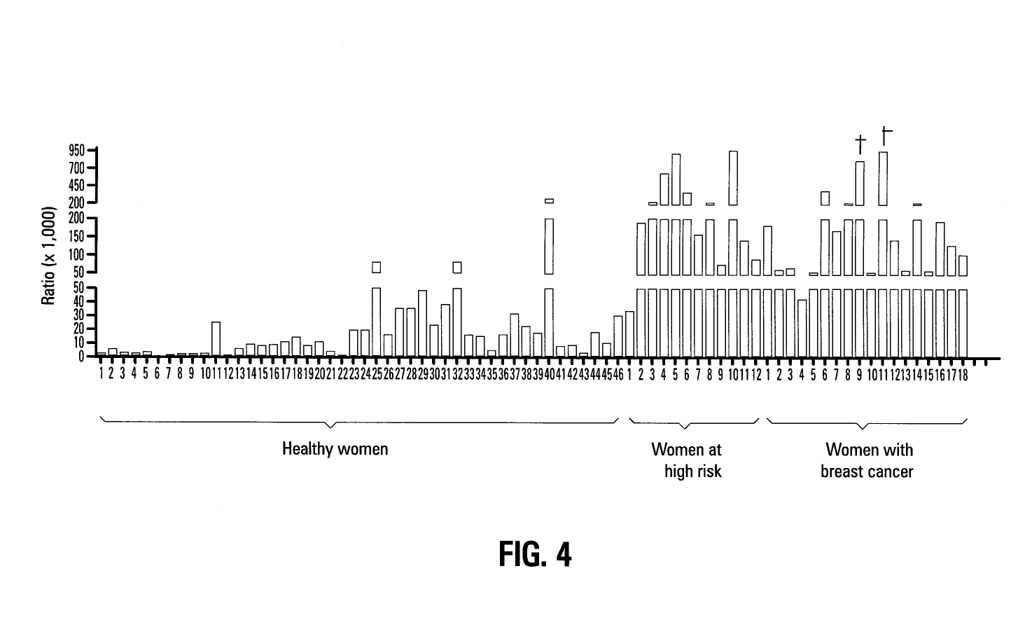

The values obtained for the various estrogen-related compounds in three groups of women were processed in two different ways. First, median values were calculated for all the compounds and their levels were examined in the three groups of women (Table 3). Then, the ratio of depurinating N3Ade and N7Gua adducts to the sum of their respective estrogen metabolites and conjugates in urine samples was used because the ratio reflects the degree of imbalance in estrogen metabolism that can lead to cancer initiation (FIG. 4). A high ratio of adducts to their respective metabolites and conjugates represents relatively more DNA damage. In contrast, a low ratio of adducts to their respective metabolites and conjugates means that relatively little of the estrogen metabolites reacted with DNA.

Median values of the urinary estrogen-related compounds in the three groups of women. Using the newly developed SPE/UPLC/MS-MS methodology, urine samples of various women's groups were analyzed for estrogen-related compounds. The data obtained were used to calculate median values for each of the 40 compounds (Table 3).

The median andostenedione, testosterone, E₂/E₁, 16α-OHE₂/16α-OHE₁, 4-OCH₃E₂/4-OCH₃E₁, 2-OHE₁(E₂) GSH conjugate and derivative values were higher for controls compared with high risk participants, and the median 4-OHE₂-1-N3Ade/4-OHE₁-1-N3Ade values were lower for controls compared with high risk participants. Compared with breast cancer participants, the median 2-OHE₂/2-OHE₁, 4-OHE₂/4-OHE₁, 16α-OHE₂/16α-OHE₁, 4-OCH₃E₂/4-OCH₃E₁, 2-OHE₁(E₂) GSH conjugate and derivative values were higher for controls, while the median 4-OHE₂-1-N7Gua/4-OHE₁-1-N7Gua values were lower for controls. Of particular interest are the significantly lower levels of the methoxycatechol estrogens in the women with breast cancer or at high risk compared to the control women, because this represents a major protective pathway in estrogen metabolism. In addition, the levels of the 4-OHE₁(E₂)-1-N3Ade and 4-OHE₁(E₂)-1-N7Gua adducts are higher in the women with breast cancer or at high risk than in the control women, although only two of the differences are statistically significant.

Depurinating estrogen-DNA adducts in the three groups of women. In the second analysis, the ratios of depurinating N3Ade and N7Gua adducts to the sum of estrogen metabolites and conjugates in urine samples from healthy control women are generally low (FIG. 4). In contrast, high ratios of these adducts to estrogen metabolites and conjugates were observed in urine from high-risk women (Gail Model score>1.66%) and women with breast carcinoma. In general, the value obtained from the high-risk women and women with breast carcinoma derives from the ratio between a high level of adducts and low levels of metabolites and conjugates. Surprisingly, in some women, the level of adducts was not particularly high, but the levels of metabolites and conjugates were very low, suggesting that a substantial proportion of the metabolites was converted to adducts. This finding indicates that for some subjects, it is important not simply to measure the absolute level of the adducts, but to calculate the ratio of the adducts to metabolites and conjugates.

In the sum of the ratios of depurinating adducts to estrogen metabolites and conjugates, the preponderant role is played by the N3Ade and N7Gua adducts of 4-OHE₁(E₂), whereas the adducts of 2-OHE₁(E₂) play a very minor role. For example, for the high-risk subject presented in Table 2, the overall adduct ratio is 936, but the contribution of 2-OHE₁(E₂)-6-N3Ade is 1, whereas the contribution of 4-OHE₁(E₂)-1-N3Ade plus 4-OHE₁(E₂)-1-N7Gua is 935. In general, the average contribution of the 2-OHE₁(E₂)-6-N3Ade adducts is approximately 2.5% of the total, whereas the predominant contribution of approximately 97.5% derives from the 4-OHE₁(E₂)-1-N3Ade and 4-OHE₁(E₂)-1-N7Gua adducts. The observation of high levels of depurinating estrogen-DNA adducts in urine from high-risk women, as well as subjects with breast carcinoma (FIG. 4), provides evidence that these adducts are a causative factor in the etiology of cancer.

Analysis by subject characteristics. The data was first analyzed using the ratio of depurinating N3Ade and N7Gua adducts to the sum of their respective estrogen metabolites and conjugates in urine samples as a continuous variable. Analysis using one-way ANOVA revealed a significant difference among the groups (p<0.001). Additional post hoc analysis using a Bonferroni correction for multiple comparisons revealed significantly higher means for high risk subjects [mean 336.45, standard deviation (SD) 331.92] compared with controls (mean 20.51, SD 37.01, p<0.001) and for breast cancer patients (mean 176.28, SD 205.68, p<0.001). The mean for patients known to be at high risk was not significantly different from that of the breast cancer group (p=0.62).

A limitation of the study is that most of the group of healthy women (forty-two of forty-six) were Italian, whereas the remaining healthy women, high-risk women and women with breast cancer were American. All of the subjects in this study were Caucasian. The three groups (healthy, high-risk and breast cancer) had similar mean age at recruitment, mean age at menarche and menopausal status (Table 4). These similarities in subject characteristics support the validity of comparing the ratios of adducts to their respective metabolites and conjugates in these three groups of women.

Subject characteristics of age at recruitment, age at menarche, menopausal status, and parity were available for 56 of the 75 subjects (Table 4). The mean age of our entirely Caucasian sample was 50 years (SD 8.5). The average age at menarche was 12.0 years (SD 1.4). Only 11% of the women were nulliparous and 43% had at least two children. Twenty-six (46%) women were premenopausal at recruitment, thirty (54%) were postmenopausal (i.e., they did not have menstrual cycles in the last 12 months before recruitment).

Analysis using one way ANOVA revealed that health status (breast cancer cases versus high risk and healthy individuals) was significantly associated with age at recruitment (p=0.048). Specifically, the mean age (years) at recruitment for healthy women was 49 (SD 7.8), 52 (SD 6.1) for women at high risk and 57 (SD 12.2) for breast cancer cases. Age at menarche was not statistically different across the disease status groups (p=0.534). Analysis using a Chi-square test did not reveal an association between health status and menopausal status (p=0.95) or parity (parous versus nulliparous) (p=0.15).

TABLE 4
Subject characteristics

| | Health Status | | | | | |
|---|---|---|---|---|---|---|
| | Healthy | | High Risk | | Breast Cancer | |
| Characteristic | n = 37 | | n = 12 | | n = 7 | |
| Age at recruitment in years, | 49 | (7.85) | 52 | (6.09) | 57 | (12.16) |
| mean (SD) | | | | | | |
| Age at menarche in years, | 12 | (1.45) | 12 | (1.44) | 13 | (1.25) |
| mean (SD) | | | | | | |

Menopausal Status, n (%)

| | | | | | | |
|---|---|---|---|---|---|---|
| Premenopausal | 17 | (46%) | 6 | (50%) | 3 | (43%) |
| Postmenopausal | 20 | (54%) | 6 | (50%) | 4 | (57%) |

Parity

| | | | | | | |
|---|---|---|---|---|---|---|
| 0 | 6 | (16%) | 0 | (%) | 0 | (0%) |
| 1 | 3 | (8%) | 0 | (0%) | 0 | (0%) |
| 2 | 13 | (35%) | 7 | (58%) | 4 | (57%) |
| 3 | 11 | (30%) | 3 | (25%) | 0 | (0%) |
| ≧4 | 4 | (11%) | 2 | (17%) | 3 | (43%) |

The correlation coefficient was used to examine the association between the ratio and subject characteristics. Evidence of significant correlation between parity and ratio (r=0.36, p=0.007) and marginally significant correlation between the ratio and menopausal status (r=0.26, p=0.06) was observed. Age at recruitment and age at menarche were not significantly associated with the ratio.

Linear regression was used to assess the association between disease status and ratio adjusted for age at recruitment, age at menarche, menopausal status (categorical) and parity for the 56 subjects with patient characteristics available (Table 5). After accounting for these characteristics, the ratio was significantly associated with health status. Specifically, the multivariate coefficient for disease status (108.6) was statistically significant (p=0.007) in a model that explained 10% (p=0.040) of variance in the ratio after accounting for covariates. All other covariates did not reach the usual level of significance of 0.05 (Table 5).

TABLE 5
Results of univariate multivariate linear regression of ratio

| | Univariate Regression | | Multivariate Regression | |
|---|---|---|---|---|
| | Regression | | Regression | |
| Covariate | Coefficient | p-value | Coefficient | p-value |
| Health status | 103.60 | 0.005 | 108.56 | 0.007 |
| Postmenopausal | 35.66 | 0.51 | 41.18 | 0.44 |
| Parity | 15.01 | 0.42 | −7.29 | 0.71 |

Interpretation of results. The discovery of high ratios of depurinating estrogen-DNA adducts to their corresponding metabolites and conjugates in urine samples from both high-risk women and women with breast cancer supports the hypothesis that formation of estrogen-DNA adducts is an initial, important step in the initiation of breast cancer. In addition, these results indicate that this assay provides a diagnostic tool for early detection of cancer risk, e.g., breast cancer risk.

In addition, the ratio of depurinating estrogen-DNA adducts to their metabolites and conjugates can be used to monitor the efficacy of putative preventive compounds for treating cancer, e.g., by balancing estrogen activation and deactivation. Minimizing formation of catechol estrogen quinones and/or their reaction with DNA should reduce the risk of developing breast cancer.

### Materials and Methods

Materials. Phenyl solid phase extraction (SPE) cartridges were purchased from Varian (Palo Alto, Calif.). Androstenedione 1, (Table 1), testosterone 2, estrone (E₁) sulfate 3, E₂ 4, E₁ 5, 2-OHE₂ 6, 2-OHE₁ 7, 16α-OHE₂ 10, 16α-OHE₁ 11, 2-OCH₃E₂ 12, 2-OCH₃E₁ 13, 4-OCH₃E₂ 14, 4-OCH₃E₁15, 2-OH-3-OCH₃E₂ 16 and 2-OH-3-OCH₃E₁ 17 were purchased from Steraloids Inc. (Newport, R.I.). 4-OHE₂ 8 and 4-OHE₁ 9 were synthesized as previously described (26), 2-OHE₂-1-SG 18, 2-OHE₂-4-SG 19, 2-OHE₁-1-SG 20, 2-OHE₁-4-SG 21, 2-OHE₂-(1+4)-Cys 22, 2-OHE₁-1-Cys 23, 2-OHE₁-4-Cys 24, 2-OHE₂-1-NAcCys 25, 2-OHE₂-4-NAcCys 26, 2-OHE₁-1-NAcCys 27, 2-OHE₁-4-NAcCys 28, 4-OHE₂-2-SG 29, 4-OHE₁-2-SG 30, 4-OHE₂-2-Cys 31, 4-OHE₁-2-Cys 32, 4-OHE₂-2-NAcCys 33 and 4-OHE₁-2-NAcCys 34 were synthesized by using the procedure of Cao et al (27), 4-OHE₂-1-N7Gua 35, 4-OHE₁-1-N7Gua 36, 4-OHE₂-1-N3Ade 37, 4-OHE₁-1-N3Ade 38, 2-OHE₂-6-N3Ade 39 and 2-OHE₁-6-N3Ade 40 were synthesized by following the reported methods (6, 7, 28). All solvents were HPLC grade and all other chemicals used were of the highest grade available.

Study population. Urine was collected from seventy-five women at three different sites: 1) at the Center for Mammographic Screening at the University of Naples, Italy (forty-two women), 2) at the Breast Diagnostic Clinic and Oncology Breast Clinic of the Mayo Clinic, Rochester, Minn. (eighteen women) and 3) at the Olson Center for Women's Health, University of Nebraska Medical Center (UNMC), Omaha, Nebr. (fifteen women). Ages ranged between 34 and 73 years. Healthy women: range, 34-67; mean, 50±8; high-risk women: range, 44-64; mean, 52±6; women with breast cancer: range, 34-73; mean, 54±10.

All women recruited at the University of Naples were healthy (i.e., they did not receive a diagnosis of breast cancer at the time of their mammographic test). Among the women recruited at the Mayo Clinic, 12 of them were classified as high-risk women (Gail Model score=1.67%-11.7%) and 6 were breast cancer cases. At UNMC, 4 women were healthy, i.e., had no known cancer, and 11 were diagnosed with breast cancer. None of the subjects received estrogen-containing treatment for at least 3 months prior to providing a urine sample. The three groups were frequency matched on age, race and menopausal status.

All procedures were approved by the University of Naples, Mayo Clinic and UNMC Institutional Review Boards.

Sample collection. A standardized method was followed to collect all of the urine samples. A spot urine sample of about 50 ml was collected from each participant and 1 mg/ml ascorbic acid was added to prevent oxidation of the catechol moieties in the various estrogen compounds. The urine samples were aliquoted, frozen and four 10-ml aliquots were transferred to the Eppley Institute, UNMC, on dry ice and were stored at −80° C. until analysis. Thus, each analytical sample was thawed only once prior to analysis.

Solid phase extraction of urine. Two milliliter aliquots of urine underwent partial purification by SPE. The SPE was performed using a 20-port SPE vacuum manifold with phenyl cartridges (FIG. 2). Urine samples were adjusted to pH 7 with 1 M NaOH or 1 M HCl. For method development and validation, 2-ml aliquots of charcoal-treated human urine samples were spiked with a total of 250, 500 or 1000 pg of the 40 estrogen-related compounds (final concentration 0.125, 0.25 and 0.50 pg/μl) and loaded onto the phenyl 100-mg cartridges pre-conditioned with CH₃OH and the loading buffer, 10 mM ammonium formate, pH 7. The cartridges were washed with the loading buffer, and then the compounds of interest were eluted from the cartridge by using an elution buffer, methanol/10 mM ammonium formate, pH 7 (90:10) with 1% acetic acid. This procedure led to enrichment of the 40 estrogen-related compounds after elution.

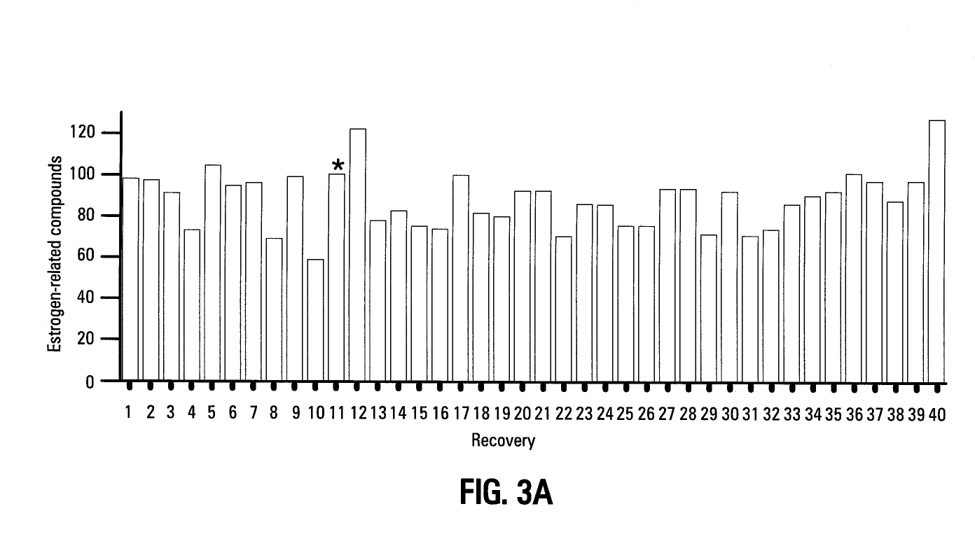

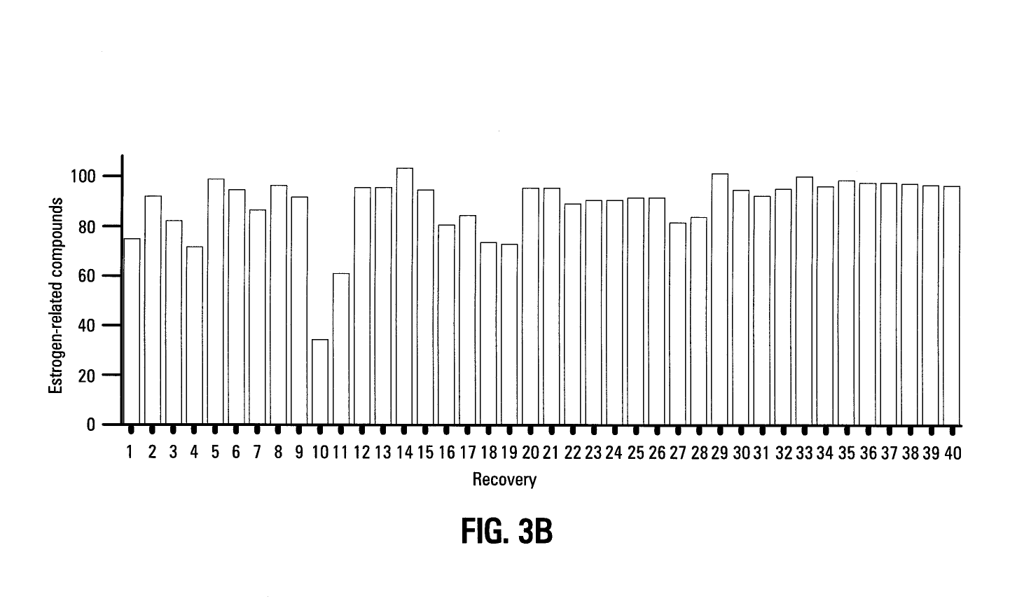

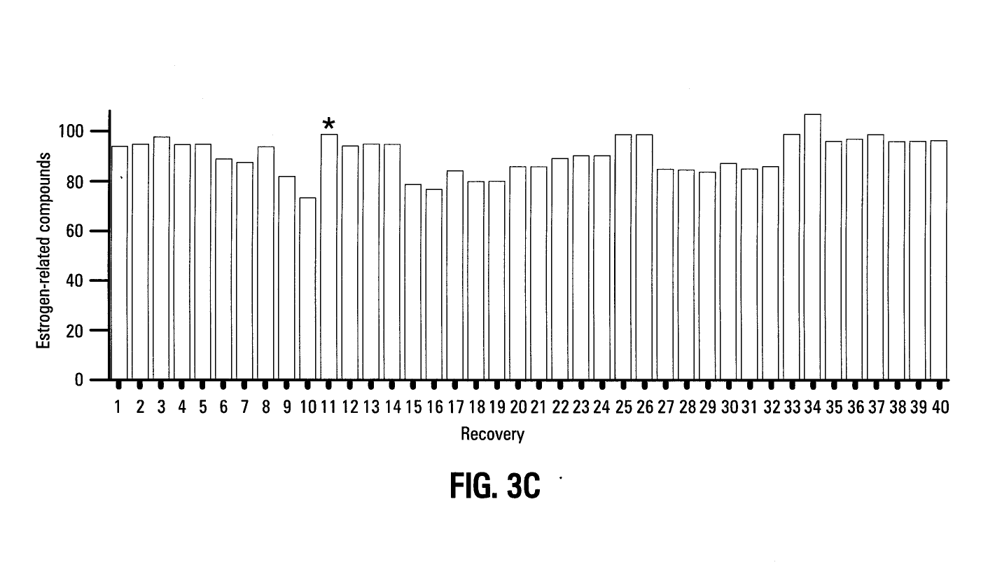

Charcoal-treated urine (2 ml) was used in controls, and the eluates from the SPE cartridges were spiked with 250, 500 or 1000 pg of the 40 estrogen-related compounds. The eluates from both the experimental and control samples were concentrated using a SpeedVac and lypholizer, and subjected to ultraperformance liquid chromatography/tandem mass spectrometry (UPLC/MS-MS) analysis. To determine the recovery of the standards by the SPE method, comparison was made between the corresponding concentrations of experimental and control samples (FIG. 3). Study samples were cleaned in duplicate by using the above optimized SPE conditions and analyzed by UPLC/MS-MS.

UPLC/MS-MS analysis of urine samples. The 40 analytes (Table 1) included the androgens androstenedione and testosterone; the estrogens E₁ sulfate, E₁ and E₂; the catechol estrogens 2-OHE₁(E₂) and 4-OHE₁(E₂); the 16α-OHE₁(E₂); the methylated 2- and 4-catechol estrogens; the 2- and 4-catechol estrogens conjugated with glutathione (GSH), cysteine (Cys) or N-acetylcysteine (NAcCys); and the depurinating DNA adducts of 4-OHE₁(E₂) and 2-OHE₁(E₂). All of the estrogen compounds were analyzed as both E₁ and E₂ derivatives because the interconversion of these two estrogens is carried out continuously by 17β-estradiol dehydrogenase.

All experiments were performed on a Waters (Milford, Mass.) Quattro Micro triple quadrupole mass spectrometer by using electrospray ionization (ESI) in positive ion (PI) and negative ion (NI) mode, with an ESI-MS capillary voltage of 3.0 kV, an extractor cone voltage of 2 V, and a detector voltage of 650 V. Desolvation gas flow was maintained at 600 l/h. Cone gas flow was set at 60 l/h. Desolvation temperature and source temperature were set to 200 and 100° C., respectively. For all the studies, a methanol:water (1:1) mixture with 0.1% formic acid was used as the carrier solution. ESI interface tuning and mass calibration were accomplished in the PI mode by using a standard sodium iodide-rubidium iodide solution. The test sample (compounds 1 through 40) was introduced to the source at a flow rate of 10 μl/min by using an inbuilt pump. PI or NI detection was used in cases where the sample was readily ionized to cation or anion, respectively. The masses of parent ion and daughter ions were obtained in the MS and MS-MS operations. The parent and daughter ion data obtained for each compound were used to generate the MRM method for UPLC/MS-MS operation (Table 1).

Measurements of estrogen-related compounds in urine extracts were conducted by using UPLC/MS-MS. UPLC/MS-MS analyses were carried out with a Waters Acquity ultraperformance liquid chromatography (UPLC) system connected with the high performance Quattro Micro triple quadrupole mass spectrometer. Analytical separations on the UPLC system were conducted using an Acquity UPLC BEH C18 1.7 μm column (1×100 mm) at a flow rate of 0.15 ml/min. The gradient started with 80% A (0.1% formic acid in H₂O) and 20% B (0.1% formic acid in CH₃CN), changed to 79% A over 4 min, followed by a 6-min linear gradient to 45% A, resulting in a total separation time of 10 min. The elutions from the UPLC column were introduced to the Quattro Micro mass spectrometer.

The ionization method used for MS analysis was ESI in both the PI and NI mode. MS-MS was performed in the MRM mode (see above), and resulting data were processed by using QuanLynx software (Waters) to quantify the estrogen metabolites. To calculate limits of detection, various concentrations, 0.1, 0.25, 0.5, 1.0, 2.5, 5.0, 10, 25, 50 and 100 pg/μl, of the analyte were injected to UPLC/MS-MS. The injected amount that resulted in a peak with a height at least two or three times as high as the baseline noise level was used as the limit of detection (Table 1). Pure standards were used to optimize the UPLC/MS conditions prior to analysis. After UPLC analysis, the mean value was calculated for all the compounds obtained from each sample.

Statistical methods. Estrogen-related compounds were compared for control vs. high risk and for control vs. breast cancer using a Mann-Whitney test, with p-values adjusted for the two multiple comparisons using the Bonferroni method. To account for the multiple hypothesis tests conducted for these variables, a p-value<0.01 was interpreted as statistically significant. The log-transformed sum of the ratios of depurinating adducts to the corresponding metabolites and conjugates was compared using a one way ANOVA, and post hoc comparisons were made using the method of Bonferroni. Linear regression was used to assess the association between disease status and ratio adjusted for age at recruitment, age at menarche, menopausal status (categorical) and parity for the 56 subjects with patient characteristics available. All the statistics and p-values were calculated using SPSS software (SPSS Inc. Chicago, Ill.).

### Example 2

### Estrogen-DNA Adducts from Men with and without Prostate Cancer

For this study, to demonstrate that the ratio of depurinating estrogen-DNA adducts to their respective metabolites and conjugates serves as a biomarker for cancers in general, 14 men with prostate cancer were studied. Their ages ranged from 50-83, with a median age of 60 and a mean age 64. Urine samples for analysis were collected at Johns Hopkins. 125 men without prostate cancer were used as controls. The age of the controls ranged from 45-78 with a median age of 67 and a mean age of 65. Urine samples for analysis for 118 of the men were collected at the University of Buffalo, and 7 samples were collected at Johns Hopkins.

The urine samples were analyzed as described in Example 1. The ratio of estrogen-DNA adducts to metabolites and conjugates for men with prostate cancer was 57.5 (median), and the ratio of estrogen-DNA adducts to metabolites and conjugates for men without prostate cancer was 23.4 (median). These ratios were statistically different (p=0.0004; p<0.01), thereby demonstrating that the ratio of depurinating estrogen-DNA adducts to their respective metabolites and conjugates serves as a biomarker for cancers in general (e.g., for breast cancer and for prostate cancer).

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While in this document the invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method for identifying a subject that has cancer, or is at an elevated risk for developing cancer, comprising determining the ratio of depurinating estrogen-DNA adducts to their respective metabolites and conjugates in a biological sample from the subject, wherein an increased level of the ratio, as compared to a control, indicates that the subject has cancer, or is at an elevated risk for developing cancer.

2. The method of claim 1, further comprising purifying the adducts, metabolites and conjugates from the sample using solid phase extraction (SPE).

3. The method of claim 2, further comprising analyzing the adducts, metabolites and conjugates utilizing ultraperformance liquid chromatography/tandem mass spectrometry (UPLC/MS-MS).

4. The method of claim 1, further comprising administering to those subjects who are at risk for developing cancer a therapy effective to reduce the risk of cancer.

5. The method of claim 4, wherein the therapy is effective to decrease the ratio of depurinating estrogen-DNA adducts to their respective metabolites and conjugates.

6. The method of claim 1, wherein the subject has, or is at an elevated risk for developing, breast cancer, prostate cancer, leukemia, non-Hodgkins lymphoma, colon cancer, pancreatic cancer, lung cancer, ovarian cancer, endometrial cancer, testicular cancer, kidney cancer or brain cancer.

7. The method of claim 1, wherein the sample is breast tissue, prostate tissue, urine, serum, sputum, nipple aspirate fluid, colon tissue, pancreas tissue, lung tissue, ovary tissue, endometrium, testis tissue, kidney tissue, or brain tissue.

8. The method of claim 1, wherein the subject is at an elevated risk for developing cancer.

9. The method of claim 1, wherein an adducts include an N3Ade adduct of 4-OHE₁(E₂) and an N7Gua adduct of 4-OHE₁(E₂).

10. The method of claim 1, wherein the subject is a male.

11. The method of claim 1, wherein the subject is a female.

12. A method for determining the effectiveness of a preventive cancer treatment, comprising determining the ratio of depurinating estrogen-DNA adducts to their respective metabolites and conjugates in a biological sample from the subject before and after a preventive cancer treatment, wherein a decrease in the level of the ratio after treatment indicates that the cancer treatment is effective to reduce the risk of cancer.

13. The method of claim 12, further comprising purifying the adducts, metabolites and conjugates from the sample using solid phase extraction (SPE).

14. The method of claim 13, further comprising analyzing the adducts, metabolites and conjugates utilizing ultraperformance liquid chromatography/tandem mass spectrometry (UPLC/MS-MS).

15. The method of claim 12, wherein the subject is at an elevated risk for developing cancer.

16. The method of claim 15, wherein the subject has, or is at an elevated risk for developing, breast cancer, prostate cancer, leukemia, non-Hodgkins lymphoma, colon cancer, pancreatic cancer, lung cancer, ovarian cancer, endometrial cancer, testicular cancer, kidney cancer or brain cancer.

17. The method of claim 12, wherein the sample is breast tissue, prostate tissue, urine, serum, sputum, nipple aspirate fluid, colon tissue, pancreas tissue, lung tissue, ovary tissue, endometrium, testis tissue, kidney tissue, or brain tissue.

18. The method of claim 12, wherein an adducts include an N3Ade adduct of 4-OHE₁(E₂) and an N7Gua adduct of 4-OHE₁(E₂).

19. The method of claim 12, wherein the subject is a male.

20. The method of claim 12, wherein the subject is a female.
